(19) United States
(12) Patent Application Publication
(10) Pub. No.: US 2012/0004522 A1
(43) Pub. Date: Jan. 5, 2012

(54) BIOSENSOR COATED WITH ELECTROACTIVE POLYMER LAYER DEMONSTRATING BENDING BEHAVIOR

(75) Inventors: Sun Ill Kim, Seoul (KR); Seon-Jeong Kim, Seoul (KR); Jang-Hyun Youn, Cypress, CA (US)

(73) Assignee: INDUSTRY-UNIVERSITY COOPERATION FOUNDATION HANYANG UNIVERSITY, Seoul (KR)

(21) Appl. No.: 13/144,324

(22) PCT Filed: Mar. 17, 2010
(86) PCT No.: PCT/KR2010/001645

§ 371 Date: Jul. 13, 2011

(30) Foreign Application Priority Data
Mar. 17, 2009 (KR) 10-2009-0022643

(51) Int. Cl. A61B 5/1473; C12M 1/34; G01N 27/00

(52) U.S. Cl. 600/345; 422/82.01; 435/287.1

(57) ABSTRACT

Disclosed is a biosensor coated with an electroactive polymer layer demonstrating a bending behavior, more specifically a biosensor including an electroactive polymer layer coated on the surface of a bioreceptor and an electrode connected to the electroactive polymer layer. When an electrical stimulation is applied to the electrode, the electroactive polymer layer shows a bending behavior and thus the surface of the bioreceptor can be exposed to an analyte to allow a concentration analysis of the analyte. When used as an implantable biosensor, the disclosed biosensor may have a substantially increased life span since the bioreceptor can be selectively exposed to the analyte.

### TECHNICAL FIELD

The present disclosure relates to a biosensor coated with an electroactive polymer layer demonstrating a bending behavior, more specifically to a biosensor including an electroactive polymer layer coated on the surface of a bioreceptor and an electrode connected to the electroactive polymer layer. When an electrical stimulation is applied to the electrode, the electroactive polymer layer shows a bending behavior and thus the surface of the bioreceptor can be exposed to an analyte to allow a concentration analysis of the analyte.

### BACKGROUND ART

Glucose, (analyte) biosensors for checking the condition of diabetic patients and preventing complications have been consistently studied over the decades and commercially available disposable sensors have been developed as a result. Although development of a sensitive implantable biosensor capable of accurately and consistently monitoring the analyte in biological systems is studied with the advance in the sensor technology, there are some obstacles.

The existing patents about glucose biosensors include, in addition to the biosensor in the most basic form for detecting electrochemical enzymatic reactions depending on the glucose concentration, a biosensor measuring the change in output caused by the change in pH or pressure due to enzymes in a hydrogel depending on the change in glucose concentration, a transdermal glucose sensor based on reverse iontophoresis, and so forth. They use different mechanisms to analyze the analyte with relatively little interruption by various complex materials existing in the blood.

However, whatever is the measurement mechanism of the biosensor, a control device capable of selectively contacting the biosensor with blood is required in order to allow the consistent measurement of the analyte concentration in the complex in complex substances such as human blood or body fluid when the biosensor is implanted in the body.

Korean Patent Nos. 541,267 and 771,711 disclose implantable biosensors allowing the consistent measurement of the analyte concentration. However, since the implantable biosensors disclosed in the patents are always exposed to the blood, proteins or other disturbing substances existing in the blood adhere onto the surface of the sensor or form films thereon. Consequently, the performance of the biosensor is degraded rapidly with time to an extent that it cannot perform as a biosensor.

### DISCLOSURE

### Technical Problem

The inventors of the present disclosure have found out that the problems of the existing implantable biosensors can be solved by attaching an electroactive polymer demonstrating a reversible volume change, especially a bending behavior, in response to an electrical stimulation by performing work with the chemical free energy in the polymer on the surface of a biosensor and then selectively applying an electrical stimulation thereto.

The present disclosure is directed to providing a biosensor including an electroactive polymer layer coated on the surface of a bioreceptor so as to allow selective operation by applying an electrical stimulation.

The present disclosure is also directed to providing an apparatus for analyzing the concentration of an analyte using an implantable biosensor that can be selectively operated.

The present disclosure is also directed to providing an implantable biosensor capable of selective operation.

The present disclosure is also directed to providing a method for selectively controlling the operation of an implantable biosensor by applying an electrical stimulation.

### Technical Solution

In one general aspect, the present disclosure provides a biosensor for analyzing the concentration of an analyte, including: a bioreceptor capable of detecting an analyte to be analyzed; a signal transducer converting a concentration information of the analyte detected by the bioreceptor to an analyzable signal; an electroactive polymer layer coated on the surface of the bioreceptor and demonstrating a bending behavior in response to an electrical stimulation; and an electrode connected to the electroactive polymer layer.

In an embodiment of the present disclosure, the electroactive polymer may be an electroactive hydrogel.

In an embodiment of the present disclosure, the surface of the electroactive polymer may be coated with a metal. Especially, the electroactive polymer may be an ionic polymer-metal composite (IPMC), and the metal may be Pt.

In an embodiment of the present disclosure, the biosensor may be an implantable biosensor.

In an embodiment of the present disclosure the analyte may be glucose.

In another general aspect, the present disclosure provides an apparatus for analyzing the concentration of an analyte using an implantable biosensor, including: a biosensor implanted in the body of a patient, the biosensor including: a bioreceptor capable of detecting an analyte to be analyzed; a signal transducer converting a concentration information of the analyte detected by the bioreceptor to an analyzable signal; an electroactive polymer layer coated on the surface of the bioreceptor and demonstrating a bending behavior in response to an electrical stimulation; and an electrode connected to the electroactive polymer layer; a means for applying an electrical stimulation to the electrode connected to the electroactive polymer layer of the biosensor; a means for transmitting a concentration analysis information generated by the biosensor; and a computer means for receiving an outputting the concentration analysis information.

In another general aspect, the present disclosure provides a method for using a biosensor implanted in the body of a patient, including: providing a biosensor including: a bioreceptor capable of detecting an analyte to be analyzed; a signal transducer converting a concentration information of the analyte detected by the bioreceptor to an analyzable signal; an electroactive polymer layer coated on the surface of the bioreceptor and demonstrating a bending behavior in response to an electrical stimulation; and an electrode connected to the electroactive polymer layer; providing a computer means connected to the biosensor and outputting the concentration value of the analyte as a data signal; implanting the biosensor in the body of a patient and applying an electrical stimulation to the electrode connected to the electroactive polymer layer of the biosensor; and receiving the concentration information of the analyte from the bioreceptor exposed to the analyte as the electroactive polymer layer demonstrates a bending behavior in response to the electrical stimulation, using the computer means, and decoding the received concentration information as the concentration value.

In another general aspect, the present disclosure provides a method for selectively controlling the operation of a biosensor implanted in the body of a patient, including: providing a biosensor including: a bioreceptor capable of detecting an analyte to be analyzed; a signal transducer converting a concentration information of the analyte detected by the bioreceptor to an analyzable signal; an electroactive polymer layer coated on the surface of the bioreceptor and demonstrating a bending behavior in response to an electrical stimulation; and an electrode connected to the electroactive polymer layer; implanting the biosensor in the body of a patient; selectively applying an electrical stimulation to the electrode connected to the electroactive polymer layer of the biosensor; and receiving the concentration information of the analyte from the bioreceptor exposed to the analyte as the electroactive polymer layer demonstrates a bending behavior in response to the selective electrical stimulation, and decoding the received concentration information as a concentration value.

### Advantageous Effects

Since the biosensor according to the present disclosure is controllable to selectively contact with an analyte using an electroactive polymer demonstrating a bending behavior, the durability and lifespan of the biosensor can be significantly enhanced.

Especially, since the contact between the surface of the biosensor and the analyte can be controlled by using the electroactive polymer demonstrating a bending behavior in response to an electrical stimulation, the problem of decreased sensor lifespan caused by the adsorption of proteins on the surface of the implantable biosensor can be solved.

### DESCRIPTION OF DRAWINGS

FIG. 1 is a perspective view showing an operation mechanism of a biosensor on which an electroactive polymer layer comprising four pieces is attached, as an embodiment of the present disclosure.

FIG. 2 is a perspective view showing an operation mechanism of a biosensor on which an electroactive polymer layer comprising two pieces is attached, as an embodiment of the present disclosure.

FIG. 3 is a cross-sectional view showing an operation mechanism of a biosensor according to an embodiment of the present disclosure.

FIG. 4 schematically shows the configuration of a biosensor according to an embodiment of the present disclosure.

### DETAILED DESCRIPTION OF MAIN ELEMENTS

A: electroactive polymer layer
S: sensor
C: channel (body fluid/blood)
PS: power supply
CC: controller

### BEST MODE

Hereinafter, the embodiments of the present disclosure will be described in detail with reference to accompanying drawings.

As used herein, “analyte” refers to a chemical constituent to be analyzed. Although biomaterials such as glucose, DNA, enzyme, protein, cell, hormone, etc. are described as examples of the analyte, general chemical substances are not excluded from the analyte.

Although a hydrogel, an interpenetrating polymer network (IPN) and an ionic polymer-metal composite (IPMC) are described as examples of an electroactive polymer in the present disclosure, those skilled in the art will understand that the present disclosure is not limited thereto but any other material capable of demonstrating a bending behavior in response to an electrical stimulation may be used.

In the present disclosure, an electroactive polymer is attached to a bioreceptor. The electroactive polymer is a material that can undergo reversible deformation in response to an external stimulation such as pH, solvent composition, on concentration, electric field, or the like. Such a system that converts a chemical free energy into a mechanical work in response to a stimulation from its surroundings is called the ‘chemomechanical system’. The electroactive polymer (EAR) is a polymer belonging to the chemomechanical system that can contract and relax or move leftward and rightward using the chemical free energy in the polymer in response to an electrical stimulation. It is a kind of polymer hydrogel.

The electroactive polymers can be classified into ones in which actuation is caused by an electric field and those in which actuation is caused by ions. The electric field-based electroactive polymers can be classified into piezoelectric, electrostrictive and ferroelectric materials. The ionization-based electroactive polymers are deformed due to displacement of ions when an electric field is applied. Polymer gel and on film are examples. Besides, various forms of electroactive polymers including carbon nanotube, paper, cloth and fluid are studied.

Because of the advantages of being reversibly deformable (contraction/relaxation or leftward/rightward movement) in response to external stimulation, having high elasticity, being lightweight and being miniaturizable, the electroactive polymer can be developed into artificial muscles, small and noiseless actuators or biosensors capable of detecting various biological signals from the living body. Thus, it is expected to bring new technical innovation in many feature industries, including robotics, biology, aviation, space, military, and microelectromechanical systems (MEMS).

In the present disclosure, an interpenetrating polymer network (IPN) hydrogel may be used as the electroactive polymer. The IPN refers to two or more networks which are at least partially interlaced on a polymer scale but not covalently bonded to each other. The network cannot be separated unless chemical bonds are broken. The IPNs are classified according to the polymerization method and type. Some IPNs form damping materials or reinforced elastomers of wide temperature range that can replace thermosetting resins. And, some IPNs exhibit continuous physical and mechanical properties that can hardly be attained with other polymers. The hydrogel refers to a network of hydrophilic polymer chains with low crosslinking density. Since it is a hydrated, crosslinked polymer system that can contain 20-90% of water in equilibrium state, it is permeable to oxygen and biocompatible. Since the IPN system is quick and sensitive to electrical stimulation and exhibits good mechanical properties (Kim at al, J. Appl. Polym. Sci., 73, 1675-1683, 1999), it can be effectively used in actuators, sensors and artificial muscles.

The electroactive polymer that may be used in the present disclosure includes any material capable of demonstrating a bending behavior in response to an electrical stimulation. Usually, a metal is coated on the polymer to enable the bending behavior in response to the electrical stimulation. The metal may be Pt, Au, Ag, Pd, Cu, etc. Usually, Pt is used in consideration of biocompatibility.

An “ionic polymer-metal composite (IPMC)” may be used as the electroactive polymer. The IPMC is a type of electroactive polymer, with metal electrodes formed on both sides of a thin polymer film. The metal electrode is usually formed by reducing metal ions. The metal may be Pt, Au, Ag, Pd, Cu, etc. However, Pt is used in general in consideration of biocompatibility.

When a voltage is applied between the two electrodes, the IPMC is bent toward the anode. Since it responds quickly and appreciably to a relatively low external voltage of 10 V or lower, it may be used to design a small, light and flexible actuator).

Generally, the IPMC is prepared by forming a metal electrode layer on an ion-Exchange polymer film that selectively passes only cations, called Nafion. It is one of the electroactive polymers that actuates when a voltage is applied. Because it is tough similarly to human muscles and can be designed to have different strengths, it is widely applicable to artificial muscles, medical sensors, etc., in addition to medical robots capable of performing medical operations while migrating between human organs.

The operation mechanism of the biosensor according to the present disclosure will be described in more detail referring to the attached drawings.

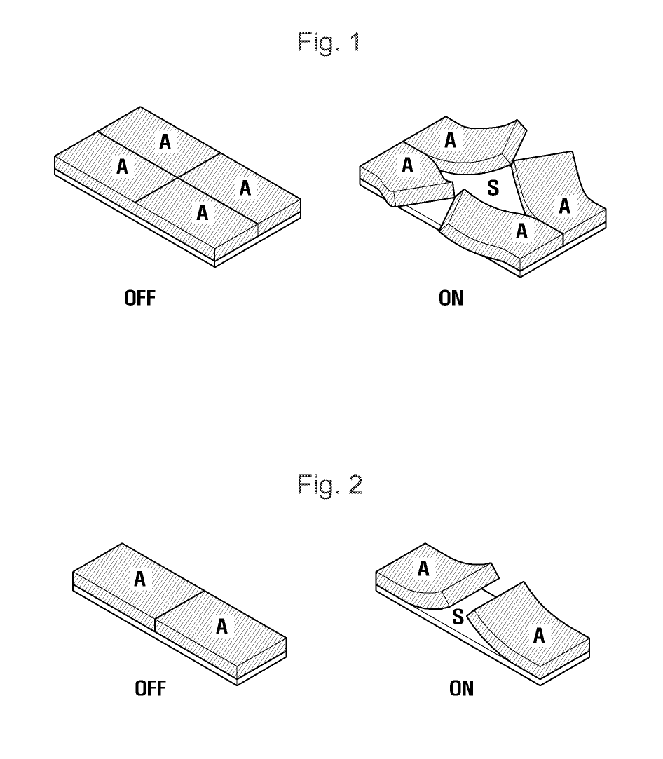

FIG. 1 is a perspective view showing an operation mechanism of a biosensor on which an electroactive polymer layer comprising four pieces is attached, as an embodiment of the present disclosure, and FIG. 2 is a perspective view showing an operation mechanism of a biosensor on which an electroactive polymer layer comprising two pieces is attached, as another embodiment of the present disclosure.

As seen from FIG. 1 and FIG. 2, when an electroactive polymer is attached to a biosensor, more specifically to a bioreceptor of the biosensor, and an electrical stimulation is applied to an electrode connected to the electroactive polymer, the electroactive polymer (electroactive hydrogel) A demonstrates a bending behavior and the surface of the biosensor S is exposed and contacted with an analyte (blood).

In FIG. 1 and FIG. 2, the OFF state on the left side is the state before applying the electrical stimulation, and the ON state on the right side is the state where the biosensor S that has been covered is exposed as the electroactive hydrogel A demonstrates a bending behavior as a result of applying the electrical stimulation. That is to say, as the electroactive hydrogel A on the surface of the biosensor S demonstrates a bending behavior in response to the electrical stimulation, the analyte (usually body fluid or blood) that has been covered by the hydrogel A is exposed to the biosensor S to allow the measurement of the analyte concentration.

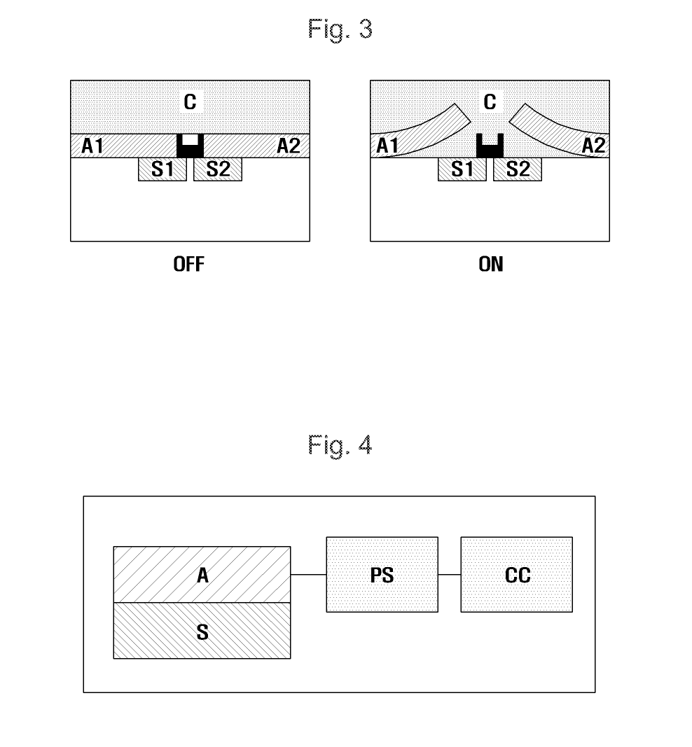

FIG. 3 is a cross-sectional view showing an operation mechanism of a biosensor according to an embodiment of the present disclosure. In FIG. 3, the OFF state on the left side is the state where an electrical stimulation is not applied and the surface of a biosensor S1, S2 is covered by an electroactive hydrogel A1, A2 without being exposed to a channel C containing an analyte, and the ON state on the right side is the state where the biosensor S1, S2 that has been covered is exposed to the channel C as the electroactive hydrogel A demonstrates a bending behavior as a result of applying an electrical stimulation.

Through this mechanism, analysis of the analyte concentration by the biosensor can be performed selectively by applying the electrical stimulation. As such, since the contact between the surface of the biosensor and the analyte can be controlled by using the electroactive polymer demonstrating a bending behavior in response to an electrical stimulation, the problem of decreased sensor lifespan caused by the adsorption of proteins on the surface of the implantable biosensor can be solved.

Consequently, even when the biosensor is implanted in the body and used for a long period of time, the problem of deteriorated function and decreased sensor lifespan caused by the adsorption of proteins on the surface of the biosensor can be solved since the time period for which and the frequency with which the bioreceptor is exposed to the analyte can be reduced.

The electroactive hydrogel A is attached to the sensor S of the implantable biosensor, and electrodes are connected to both ends of the electroactive hydrogel A in order to apply an electrical stimulation to the electroactive hydrogel A. The electrode may comprise an unharmful biocompatible material, and may have a shape of needle, plate or disc. The electrode may comprise a single electrode or multiple electrodes arranged in array form attached or fixed to the hydrogel so as to apply the electrical stimulation.

FIG. 4 schematically shows how the operation of an implantable biosensor according to an embodiment of the present disclosure is controlled using a controller. A power supply PS supplies power to an electroactive hydrogel A via an electrode, and a controller CC generates a power control signal and transmits it to the power supply PS for analysis of an analyte. When the power is turned off, the electroactive hydrogel A is set to such a position that the biosensor S is not exposed to a channel C for protection of the sensor.

For instance, when it is desired to analyze an analyte using the implantable biosensor S, the controller CC generates a signal for controlling the electroactive hydrogel A and transmits it to the power supply PS. Then, the power supply PS supplies the power for controlling the electroactive hydrogel A. When the power is supplied in response to the control signal, the electroactive hydrogel A is bent and, as a result, the biosensor S is allowed to selectively contact with the analyte in the channel C.

The control signal from the implantable biosensor may be automatically transmitted to the controller CC of the biosensor. In response to the control signal, the controller CC may transmit an operation signal to the power supply PS, thus allowing the control of power supply to the power supply PS and measurement of analyte concentration by the biosensor. Through such a control action, the biosensor may be allowed to contact with the blood or body fluid only for a minimum time period when the measurement is desired.

The biosensor according to an embodiment of the present disclosure may be provided as an apparatus for analyzing the concentration of an analyte together with a means for applying an electrical stimulation to the electrode connected to the electroactive polymer of the biosensor, a means for transmitting the concentration analysis information generated by the biosensor, and a computer means for receiving the concentration analysis information from the means for transmitting the information and outputting it.

A method for using an implantable biosensor with an electroactive polymer layer demonstrating a bending behavior attached implanted in the body of a patient is as follows.

A biosensor coated with an electroactive polymer layer demonstrating a bending behavior is provided, and a computer means connected to the biosensor and outputting a concentration value of an analyte as a data signal is provided. The biosensor is implanted in the body of a patient and an electrical stimulation is applied to an electrode connected to the electroactive polymer layer. When the electroactive polymer layer demonstrates a bending behavior in response to the applied electrical stimulation, the bioreceptor is exposed to the analyte and the concentration information of the analyte detected by the bioreceptor is received via the computer means. The received concentration information may be decoded as a concentration value.

The biosensor according to an embodiment of the present disclosure may be selectively controlled by applying an electrical stimulation. Details are as follows.

A biosensor coated with an electroactive polymer layer demonstrating a bending behavior is provided, and the biosensor is implanted in the body of a patient. Then, an electrical stimulation is selectively applied to an electrode connected to the electroactive polymer layer. When the electroactive polymer layer demonstrates a bending behavior in response to the applied electrical stimulation, the bioreceptor is exposed to the analyte and the concentration information of the analyte detected by the bioreceptor is received. The received concentration information may be decoded as a concentration value.

Those skilled in the art will appreciate that the conceptions and specific embodiments disclosed in the foregoing description may be readily utilized as a basis for modifying or designing other embodiments for carrying out the same purposes of the present disclosure. Those skilled in the art will also appreciate that such equivalent embodiments do not depart from the spirit and scope of the disclosure as set forth in the appended claims.

1. A biosensor for analyzing the concentration of an analyte, comprising:
a bioreceptor capable of detecting an analyte to be analyzed;
a signal transducer converting a concentration information of the analyte detected by the bioreceptor to an analyzable signal;
an electroactive polymer layer coated on the surface of the bioreceptor and demonstrating a bending behavior in response to an electrical stimulation; and
an electrode connected to the electroactive polymer layer.

2. The biosensor according to claim 1, wherein the electroactive polymer is an electroactive hydrogel.

3. The biosensor according to claim 1, wherein the surface of the electroactive polymer is coated with a metal.

4. The biosensor according to claim 3, wherein the electroactive polymer is an ionic polymer-metal composite (IPMC).

5. The biosensor according to claim 3, wherein the metal is Pt.

6. The biosensor according to claim 1, wherein the biosensor is an implantable biosensor.

7. The biosensor according to claim 1, wherein the analyte is glucose.

8. An apparatus for analyzing the concentration of an analyte using an implantable biosensor, comprising:
a biosensor implanted in the body of a patient, the biosensor comprising: a bioreceptor capable of detecting an analyte to be analyzed; a signal transducer converting a concentration information of the analyte detected by the bioreceptor to an analyzable signal; an electroactive polymer layer coated on the surface of the bioreceptor and demonstrating a bending behavior in response to an electrical stimulation; and an electrode connected to the electroactive polymer layer;
a means for applying an electrical stimulation to the electrode connected to the electroactive polymer layer of the biosensor;
a means for transmitting a concentration analysis information generated by the biosensor; and
a computer means for receiving an outputting the concentration analysis information.

9. A method for using a biosensor implanted in the body of a patient, comprising:
providing a biosensor comprising: a bioreceptor capable of detecting an analyte to be analyzed; a signal transducer converting a concentration information of the analyte detected by the bioreceptor to an analyzable signal; an electroactive polymer layer coated on the surface of the bioreceptor and demonstrating a bending behavior in response to an electrical stimulation; and an electrode connected to the electroactive polymer layer;
providing a computer means connected to the biosensor and outputting the concentration value of the analyte as a data signal;
implanting the biosensor in the body of a patient and applying an electrical stimulation to the electrode connected to the electroactive polymer layer of the biosensor; and
receiving the concentration information of the analyte from the bioreceptor exposed to the analyte as the electroactive polymer layer demonstrates a bending behavior in response to the electrical stimulation, using the computer means, and decoding the received concentration information as the concentration value.

10. A method for selectively controlling the operation of a biosensor implanted in the body of a patient, comprising:
providing a biosensor comprising: a bioreceptor capable of detecting en analyte to be analyzed; a signal transducer converting a concentration information of the analyte detected by the bioreceptor to an analyzable signal; an electroactive polymer layer coated on the surface of the bioreceptor and demonstrating a bending behavior in response to an electrical stimulation; and an electrode connected to the electroactive polymer layer;
implanting the biosensor in the body of a patient;
selectively applying an electrical stimulation to the electrode connected to the electroactive polymer layer of the biosensor; and
receiving the concentration information of the analyte from the bioreceptor exposed to the analyte as the electroactive polymer layer demonstrates a bending behavior in response to the selective electrical stimulation, and decoding the received concentration information as a concentration value.
